(19) United States
(12) Patent Application Publication
(10) Pub. No.: US 2016/0346544 A1
(43) Pub. Date: Dec. 1, 2016

(54) SELF-CONTAINED, HANDHELD BIPOLAR CORTICAL STIMULATOR

(71) Applicant: RUSH UNIVERSITY MEDICAL CENTER, Chicago, IL (US)

(72) Inventor: Richard W. BYRNE, Oak Brook, IL (US)

(21) Appl. No.: 15/117,397

(22) PCT Filed: Feb. 6, 2015
(86) PCT No.: PCT/US15/14777

§ 371 Date: Aug. 8, 2016

Related U.S. Application Data
(60) Provisional application No. 61/938,456, filed on Feb. 11, 2014.

(51) Int. Cl. A61N 1/36; A61B 5/00; A61N 1/04; A61B 5/0476

(52) U.S. Cl. CPC A61N 1/36025; A61B 5/0476; A61B 5/4094; A61N 1/0456; A61N 1/0472

(57) ABSTRACT

A bipolar cortical stimulator comprises a handheld housing including one or more batteries for powering the bipolar cortical stimulator and stimulator element extending from the housing. The bipolar cortical stimulator further includes a circuit board disposed within the housing for controlling operation of the stimulator element and powered by the one or more batteries. A plurality of controls extend from the housing and are operatively connected to the circuit board to control one or more parameters of the stimulator element. A digital display is disposed within the housing for displaying the one or more parameters of the stimulator element.

### BACKGROUND

Bipolar cortical stimulators and more particularly, bipolar cortical stimulators that are self-contained, handheld, and portable, are disclosed. These stimulators are useful for stimulating cortex or subcortical white matter of the human brain.

Bipolar cortical stimulators are used to stimulate cortex or subcortical white matter of the human brain in order to identify functional cortex, such as motor or speech cortex. There are many disadvantages to current bipolar cortical stimulators, for example, they are cumbersome and they require at least two people to separately control the stimulator and the power unit.

### SUMMARY

Bipolar cortical stimulator apparatuses are disclosed herein. Such bipolar cortical stimulators are self-contained, handheld, and portable to allow a surgeon to move freely throughout a surgical field and further allows the surgeon to operate the bipolar cortical stimulator and to simultaneously change parameters of the bipolar cortical stimulator.

A single-unit handheld bipolar cortical stimulator may be battery operated, may have a digital display, and may be sterilizable or covered. A self-contained handheld unit makes the process of stimulating the cortex of a human more efficient, by saving money and time. This brings the stimulator under the surgeon's control to improve safety and efficiency and shorten the time to perform cortical mapping.

In illustrative embodiments, a bipolar cortical stimulator may include a handheld housing including one or more batteries for powering the bipolar cortical stimulator, and a stimulator element extending from the housing. The stimulator may further include a circuit board disposed within the housing for controlling operation of the stimulator element and powered by the one or more batteries, and a plurality of controls extending from the housing and operatively connected to the circuit board to control one or more parameters of the stimulator element. Functions of controls include allowing an increase in duration, allowing a decrease in duration, allowing an increase in frequency, allowing a decrease in frequency, allowing an increase in current, and allowing a decrease in current.

Still further, the stimulator may include a digital display disposed within the housing for displaying the one or more parameters of the stimulator element.

In any of the embodiments herein, the stimulator may include a sterilizable cover disposed over and covering a housing. In any of the embodiments herein, the sterilizable cover may be disposable. In any of the embodiments herein, the sterilizable cover may cover at least a portion of the stimulator element. In any of the embodiments herein, the stimulator may include a digital display that includes a first display for displaying a current or selected duration, a second display for displaying a current or selected frequency, and a third display for displaying a current or selected current.

In any of the embodiments herein, the stimulator may further comprise an electroencephalograph.

In any of the embodiments herein, the electroencephalograph may be operatively connected to the circuit board and may record electrical activity from the stimulator element.

In any of the embodiments herein, the electroencephalograph may be configured to record electrical activity when current exerted by the bipolar cortical stimulator across the stimulator element is reduced to about 0 milliamps.

In any of the embodiments herein, the stimulator may comprise a digital electroencephalogram display disposed within the housing for displaying an electroencephalogram recorded by the electroencephalograph.

In any of the embodiments herein, the stimulator may comprise an additional controller extending from the housing and operatively connected to the circuit board to allow the bipolar cortical simulator to switch between a cortical stimulating mode by turning the stimulator element on and an electroencephalogram recording mode by turning the stimulator element off.

In any of the embodiments herein, the stimulator may comprise a seizure warning element operatively connected to the circuit board and configured to convey a warning when a single recorded electroencephalogram or a series of recorded electroencephalograms provide an inference of a seizure.

In an illustrative embodiment, a bipolar cortical stimulator may include a handheld housing including one or more batteries for powering the bipolar cortical stimulator and a stimulator element extending from the housing. The stimulator may further include a circuit board disposed within the housing for controlling operation of the stimulator element and powered by the one or more batteries and a control extending from the housing and operatively connected to the circuit board to control an increase in current or a decrease in current of the stimulator element.

In illustrative embodiments, a method of stimulating cortex in a patient may include the steps of holding the housing of the bipolar cortical stimulator with a first hand of a user, placing the stimulator element adjacent the cortex of the patient, and operating the plurality of controls with a second hand of the user to change the one or more parameters of the stimulator element. In other illustrative embodiments, the method may further include the step of monitoring the electroencephalogram of the patient for a sign of a seizure.

In any of the embodiments herein, a method of stimulating cortex in a patient may include the step of reading the plurality of controls and simultaneously operating the plurality of controls to further change the one or more parameters of the stimulator element.

### BRIEF DESCRIPTION OF THE DRAWINGS

FIG. 1 depicts a control unit for a prior art bipolar cortical stimulator;

FIG. 2 depicts the control unit of FIG. 1 and an attached prior art bipolar cortical stimulator at use in an operating room; and

FIG. 3 depicts a portable, handheld bipolar cortical stimulator of the present disclosure;

FIG. 4 depicts a further embodiment of a portable, handheld bipolar cortical stimulator of the present disclosure.

FIG. 5 depicts a further embodiment of a portable, handheld bipolar cortical stimulator of the present disclosure, showing a sterilizable cover.

FIG. 6 depicts a further embodiment of a portable, handheld bipolar cortical stimulator of the present disclosure, showing a display for an electroencephalogram.

FIG. 7 depicts a further embodiment of a portable, handheld bipolar cortical stimulator of the present disclosure, showing a display for an electroencephalogram and a sterilizable cover.

Other aspects and advantages of the present disclosure will become apparent upon consideration of the following detailed description, wherein similar structures have like or similar reference numerals.

### DETAILED DESCRIPTION

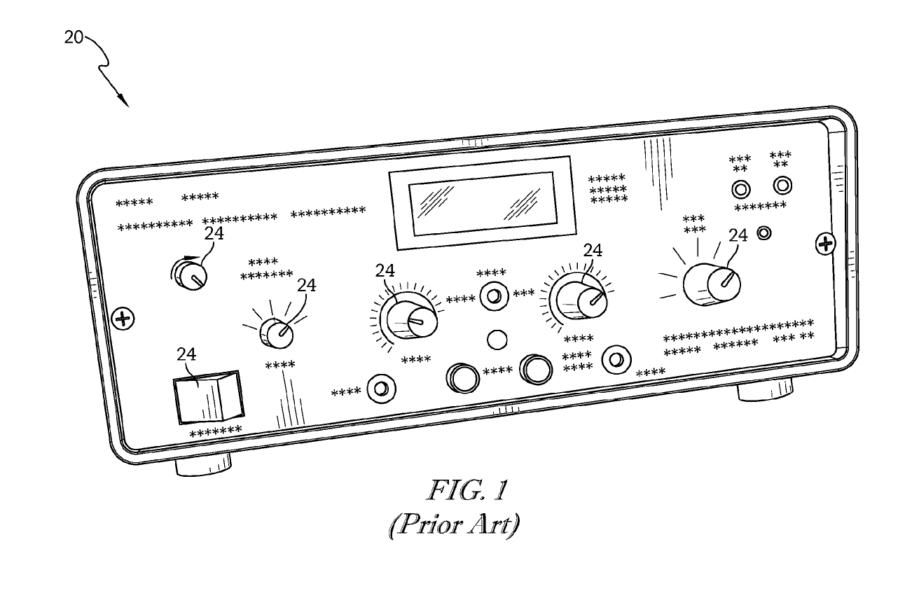

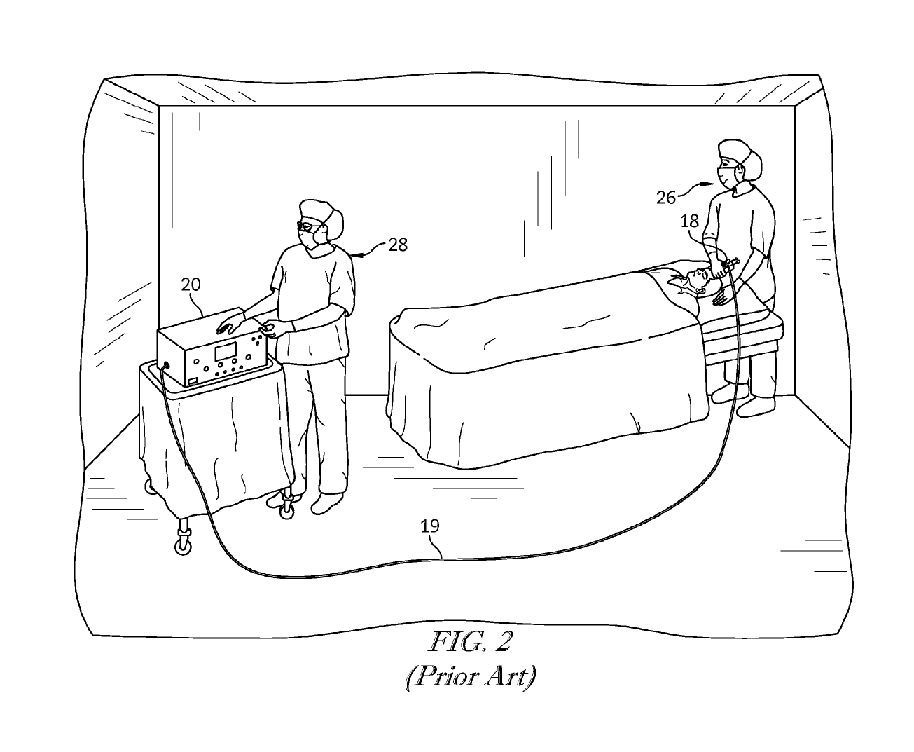

As seen in FIGS. 1 and 2, current bipolar cortical stimulators include a stimulator 18 that is connected by a cord 19 to a power unit 20. The power unit 20 includes any number of buttons 22 and knobs 24 to control the operation of the bipolar cortical stimulator and to turn the power unit 20 on and off. The power unit 20 is not sterile, so it must be located outside the sterile operating field, thereby requiring a large distance between a surgeon or other healthcare professional 26 operating the stimulator 18 and the power unit 20. The stimulator 18 is, therefore, connected by the cord 19, which usually extends across the operating room, to the power unit 20. As depicted in FIG. 2, during use of the bipolar cortical stimulator 18, a first healthcare professional, namely, the surgeon 26, operates the bipolar cortical stimulator 18 and a second healthcare professional, namely a circulating nurse 28, operates the power unit 20, which can cause delays and potential errors. If the circulating nurse is operating the power unit 20, this distracts the circulating nurse from his or her normal duties in the operating room and also makes the process inefficient and slow. Further, if the circulating nurse does not operate the power unit 20, an additional person may be necessary in the operating room, thereby increasing costs.

The present disclosure is directed to bipolar cortical stimulators and methods of using the same. While the apparatuses and methods of the present disclosure may be embodied in many different forms, several specific embodiments are discussed herein with the understanding that the present disclosure is to be considered only as an exemplification of the principles of the disclosure, and it is not intended to limit the disclosure to the embodiments illustrated.

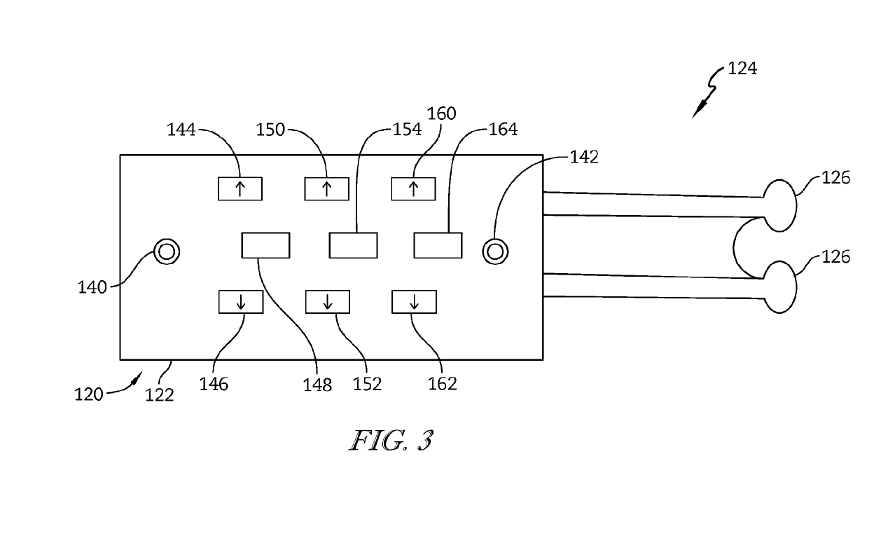

Referring to FIG. 3, an illustrative bipolar cortical stimulator 120 of the present disclosure is depicted. The bipolar cortical stimulator 120 is a portable, handheld unit that may be easily moved throughout a surgical field. In particular, the bipolar cortical stimulator 120 includes a power unit (not shown) that supplies power to, allows for operation of, and which is incorporated within the handheld bipolar cortical stimulator 120. More specifically, the bipolar cortical stimulator 120 includes an internal power source, for example, in the form of batteries (not shown) that power the bipolar cortical stimulator 120. In this manner, a surgeon operating the bipolar cortical stimulator 120 may change the various parameters of the stimulator 120, as will be described in detail below, as he or she is using the stimulator 120 on a patient.

As seen in FIG. 2, the bipolar cortical stimulator 120 generally includes a housing 122 for holding batteries (or any other suitable power source), a cortical stimulator element 124 extending from a side of the housing 122, and circuitry disposed within the housing 122 for operating the stimulator element 124. The circuitry may be in the form of an application specific integrated circuit (ASIC), microcontroller, or any other suitable circuitry. As will be discussed in more detail below, the circuitry controls operation of the stimulator element 124. The stimulator element 124 includes bipolar tips 126 for stimulating the cortex of a patient.

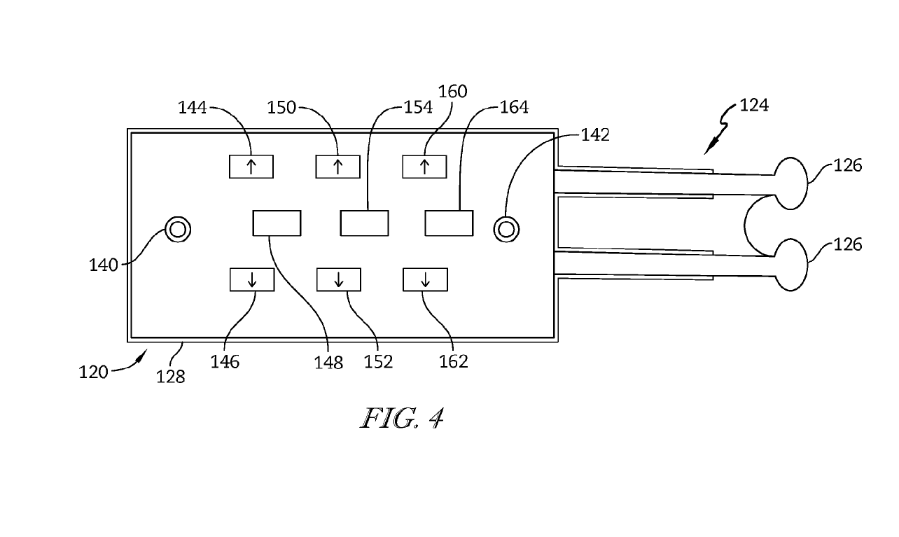

The bipolar cortical stimulator 120 may include a sterilizable cover 128, as seen in FIG. 4. The sterilizable cover 128 may cover the housing 122 and/or portions of the stimulator element 124 to create a sterile instrument. The cover 128 may be disposable or may be re-usable and sterilized between uses. In an illustrative embodiment, the cover 128 is clear and allows a user to see and activate the controls on the housing 122. In other illustrative embodiments, only portions of the cover 128 overlying controls on the housing 122 may be clear. The cover 128 may be made of plastic, cloth, or any other sterilizable material.

A number of controls, for example, in the form of buttons, slide switches, or any other suitable types of controls may extend from the housing 122 to control the stimulator element 124. The housing 122 may also include a number of digital displays for displaying various features and/or operational characteristics of the bipolar cortical stimulator 120.

In an illustrative embodiment, the housing 122 includes a control 140 to turn the bipolar cortical stimulator 120 on and off and a control 142 to turn the stimulator element 124 on and off The bipolar cortical stimulator 120 may also include controls 144, 146 to increase and decrease a duration of application of a pulse from the stimulator element 124 and a duration digital display 148 to display a selected application time (for example, in seconds). The duration digital display 148 may be configured to display the current duration, such that, as a user is using the controls 144, 146 to vary the duration, the duration on the duration digital display 148 changes with the user's selections.

The bipolar cortical stimulator 120 may further include controls 150, 152 to increase or decrease a frequency of the stimulator element 124 and a frequency digital display 154 to display a selected frequency (for example, in hertz). The frequency digital display 154 may be configured to display the current frequency, such that, as a user is using the controls 150, 152 to vary the frequency, the frequency on the frequency digital display 154 changes with the user's selections.

The bipolar cortical stimulator 120 may further include controls 160, 162 to increase or decrease a current applied to the stimulator element 124 and a current digital display 164 to display a selected current (for example, in milliamps). The current digital display 164 may be configured to display the current or selected current, such that, as a user is using the controls 160, 162 to vary the current, the current on the current digital display 164 changes with the user's selections.

While the controls 144, 146, 150, 152, 160, and 162 are depicted as buttons, such controls may optionally be implemented as other types of controls. In an illustrative embodiment, one or more of the sets of controls used to increase and decrease an operational parameter may be implemented by a single slide switch that allows a user to increase and decrease that operational parameter. Still further, any other alternative or additional controls may be utilized.

The circuitry disposed within the housing 122 is configured to control operation of the stimulator 124 and bipolar tips 126. In particular, the circuitry senses or receives input from the controls 140, 142, 144, 146, 150, 152, 160, 162. If activation of one of the controls 140, 142 is sensed or notification thereof is received, the circuitry turns the stimulator 120 or stimulator element 124, respectively, on or off. If input is received or sensed from the controls 144, 146, the circuitry increases or decreases the duration for application and changes the duration on the screen 148 accordingly. Similarly, if input is received or sensed from the controls 150, 152, the circuitry increases or decreases the frequency and changes the frequency on the screen 154 accordingly. In addition, if input is received or sensed from the controls 160, 162, the circuitry increases or decreases the current and changes the current on the screen 164 accordingly.

The circuitry of the bipolar cortical stimulator 120 may communicate wirelessly with other systems, for example, a computer, a PDA, or a tablet to download, display, or otherwise use real time data received from the bipolar cortical stimulator 120. In this manner, data regarding use of the bipolar cortical stimulator 120 may be stored in, for example, a database and later accessed by the surgeon. Still further, the data may be simultaneously monitored on another system, either in the operating room or remotely.

While the bipolar cortical stimulator 120 is described as having batteries, the bipolar cortical stimulator 120 may optionally include any other internal power source. Still optionally, the batteries may be rechargeable batteries.

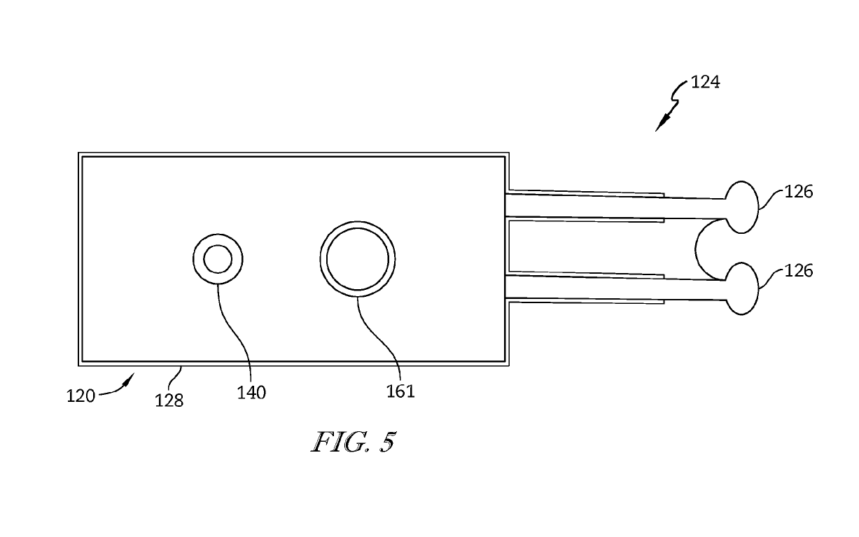

In a further embodiment, as shown in FIG. 5, the cortical stimulator 120 may be manufactured inexpensively for disposability. In particular, the cortical stimulator 120 could be designed with an on/off control 140 and a dial 161 for adjusting the current (for example, in milliamps). In such an embodiment, the frequency, pulse duration, and other values would be fixed. The simplicity of the device would allow the device to be portable and disposable, in which case, a different cortical stimulator 120 could be used for each new patient or procedure.

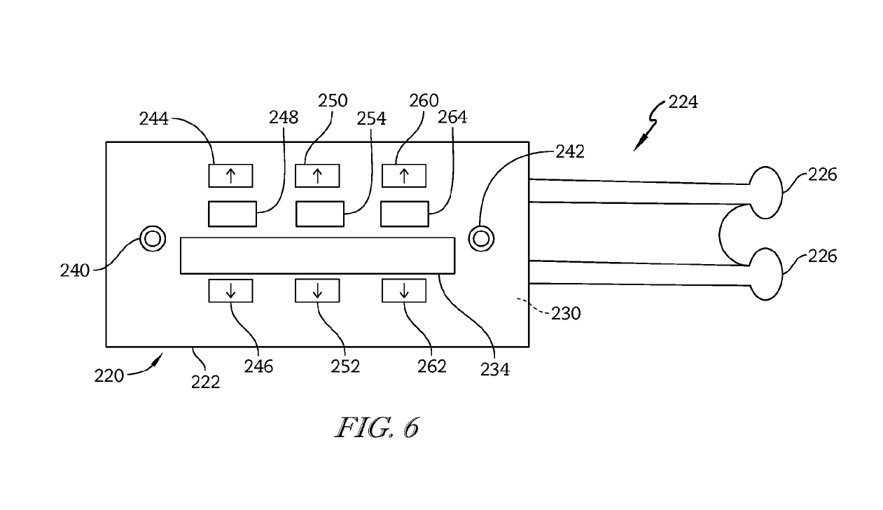

In a further embodiment, as shown in FIG. 6, a bipolar cortical stimulator 220 includes a housing 222 for holding batteries (or any other suitable power source), a cortical stimulator element 224 extending from a side of the housing 222 and having a pair of bipolar tips 226, an electroencephalograph 230, and circuitry disposed within the housing 222 for operating the stimulator element 224 and the electroencephalograph 230. The electroencephalograph 230 may record electrical activity between the bipolar tips 226 of the cortical stimulator element 224. As will be discussed in further detail below, the bipolar cortical stimulator 220 may include a plurality of controls 240, 242, 244, 246, 250, 252, 260, and 262 similar to the controls of the bipolar cortical stimulator 120 of FIGS. 3 and 4. Similarly, also discussed below, the bipolar cortical stimulator 220 may include a plurality of displays 248, 254, and 264 similar to the controls of the bipolar cortical stimulator 120 of FIGS. 3 and 4.

In an illustrative embodiment, when the stimulator element 224 is turned off, the electroencephalograph 230 may record an electroencephalogram and displays the electroencephalogram on an electroencephalogram display 234. When the stimulator element 224 is turned off, current exerted by the bipolar cortical stimulator 220 across the stimulator element 224 is reduced to about 0 milliamps. When the stimulator element 224 is turned on, the electroencephalograph 230 may stop recording an electroencephalogram. When the stimulator element 224 is turned off again, the electroencephalogram 230 may record another electroencephalogram. The electroencephalogram display 234 may show a space between recorded electroencephalograms.

The electroencephalograms displayed by the electroencephalogram display 234 may be examined to determine if a patient has suffered a seizure. When the bipolar cortical stimulator 220 is in the cortical stimulating mode, the bipolar cortical stimulator 220 gives an electrical impulse limited to the space between a pair of electrode balls that contact a surface of a brain. The pair of electrode balls are contacted by the bipolar tips 226 of the stimulator element 224. Ideally the physiological response is limited to the space being stimulated between the electrode balls. This may result in an afterdischarge, a simple partial seizure caused by the stimulation. In an afterdischarge, there is regional spread of the electrical impulse to the surrounding brain that continues on after the impulse has stopped. The afterdischarge may not be limited to the space between the pair of electrode balls and any physiologic response generated when the afterdischarge occurs may represent a regional effect and not a local effect. The afterdischarge may lead to a generalized seizure, which can be dangerous in an awake patient. The afterdischarge is simple, regular and rhythmic, lasting anywhere from seconds to minutes. When the bipolar cortical stimulator 220 is in the electroencephalogram recording mode, a neurosurgeon may identify the afterdischarge by reviewing the electroencephalogram display 234.

The bipolar cortical stimulator 220 may include a seizure warning element. The circuitry disposed within the housing 222 may be configured to detect an afterdischarge and turn on the seizure warning element. The seizure warning element may alert the neurosurgeon through light, sound, vibration, a combination, or any other method. The circuity disposed within the housing 222 may be configured based on algorithms created by epileptologists that pick up simple partial seizures, including an afterdischarge.

The housing 222 may include a control 240 to turn the bipolar cortical stimulator 220 on and off and/or a control 242 to turn the stimulator element 224 on and off. The bipolar cortical stimulator 220 may also include the controls 244, 246 to increase and decrease a duration of application of a pulse from the stimulator element 224 and a duration digital display 248 to display a selected application time (for example, in seconds). The duration digital display 248 may be configured to display the current duration, such that, as a user is using the controls 244, 246 to vary the duration, the duration on the duration digital display 248 changes with the user's selections.

The bipolar cortical stimulator 220 may further include the controls 250, 252 to increase or decrease a frequency of the stimulator element 224 and a frequency digital display 254 to display a selected frequency (for example, in hertz). The frequency digital display 254 may be configured to display the current frequency, such that, as a user is using the controls 250, 252 to vary the frequency, the frequency on the frequency digital display 254 changes with the user's selections.

The bipolar cortical stimulator 220 may further include the controls 260, 262 to increase or decrease a current applied to the stimulator element 224 and a current digital display 264 to display a selected current (for example, in milliamps). The current digital display 264 may be configured to display the current or selected current, such that, as a user is using the controls 260, 262 to vary the current, the current on the current digital display 264 changes with the user's selections.

While the controls 244, 246, 250, 252, 260, and 262 are depicted as buttons, such controls may optionally be implemented as other types of controls. In an illustrative embodiment, one or more of the sets of controls used to increase and decrease an operational parameter may be implemented by a single slide switch that allows a user to increase and decrease that operational parameter. In another embodiment, one or more of the sets of controls used to increase and decrease an operational parameter may be implemented by a dial or dials that allows a user to increase and decrease that operational parameter. Still further, any other alternative or additional controls may be utilized.

When the bipolar cortical stimulator 220 is on, it may be in a stimulation mode or a recording mode. The bipolar cortical stimulator 220 is in the stimulation mode when the stimulator element 224 is on. The bipolar cortical stimulator 220 is in the recording mode when the stimulator element 224 is off.

The circuitry disposed within the housing 222 is configured to control operation of the stimulator element 224 and the bipolar tips 226. In particular, the circuitry senses or receives input from the controls 240, 242, 244, 246, 250, 252, 260, 262. If activation of one of the controls 240, 242 is sensed or notification thereof is received, the circuitry turns the bipolar cortical stimulator 220 or the stimulator element 224, respectively, on or off. If the stimulator element 224 is on, the bipolar cortical stimulator 220 is in a stimulation mode. If input is received or sensed from the controls 244, 246, the circuitry increases or decreases the duration for application and changes the duration on the screen 248 accordingly. Similarly, if input is received or sensed from the controls 250, 252, the circuitry increases or decreases the frequency and changes the frequency on the screen 254 accordingly. In addition, if input is received or sensed from the controls 260, 262, the circuitry increases or decreases the current and changes the current on the screen 264 accordingly.

When the stimulator element 224 is turned off, the electroencephalograph 230 is turned on, and the bipolar cortical stimulator 220 is in the recording mode. When the bipolar cortical stimulator is in the recording mode, the electroencephalograph 230 may record an electroencephalogram and displays the electroencephalogram on an electroencephalogram display 234. When the stimulator element 224 is turned on, the electroencephalograph 230 may stop recording an electroencephalogram. When the stimulator element 224 is turned off again, the electroencephalogram 230 may record another electroencephalogram. The electroencephalogram display 234 may show a space between recorded electroencephalograms.

The circuitry disposed within the housing 222 may be further configured to control operation of the electroencephalograph 230, the bipolar tips 226, and the electroencephalogram display 234. In particular, the circuitry senses or receives input from the controls 240, 242. If activation of one of the controls 240, 242 is sensed or notification thereof is received, the circuitry turns the bipolar cortical stimulator 220 or the stimulator element 224, respectively, on or off. If the bipolar cortical stimulator 220 is on and the stimulator element 224 is off, the bipolar cortical stimulator 220 is in a recording mode. The circuitry is configured to connect the bipolar tips 226 of the stimulation element 224 to the electroencephalograph 230 so that the electroencephalograph 230 may record electrical activity between the bipolar tips 226. The circuitry is configured to connect the electroencephalograph 230 and the electroencephalogram display 234 so that the electrical activity between the bipolar tips 226 is translated to an electroencephalogram displayed on the electroencephalogram display 234. If the bipolar cortical stimulator 220 is switched to stimulation mode, the circuitry stops the electroencephalograph 230 from recording the electrical activity between the bipolar tips 226 as an electroencephalogram on the electroencephalogram display 234. When the bipolar cortical stimulator 220 is switched back to recording mode, the electroencephalograph 230 may resume recording the electrical activity between the bipolar tips as a subsequent electroencephalogram on the electroencephalogram display 234. The circuitry may control the electroencephalogram display 234 such that the subsequent encephalogram is spaced apart from the prior encephalogram. Alternatively, the circuitry may control the electroencephalogram display 234 such that the subsequent encephalogram is not spaced apart from the prior encephalogram.

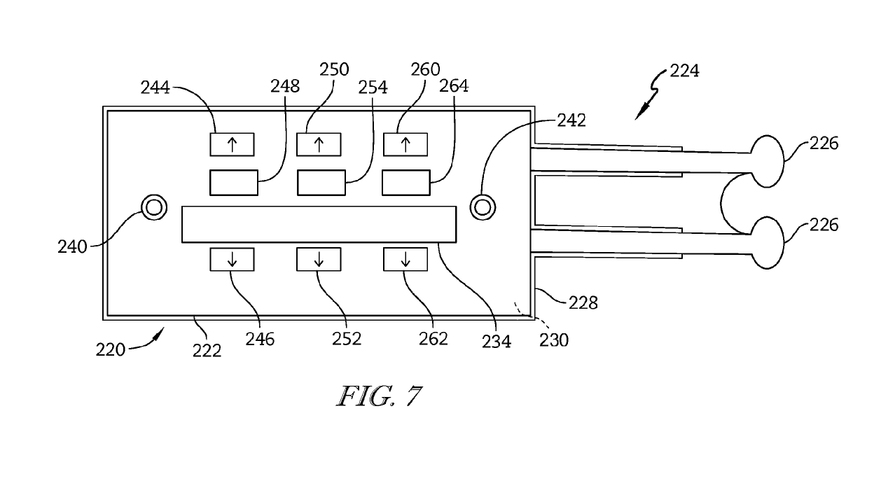

The bipolar cortical stimulator 220 may include a sterilizable cover 228, as seen in FIG. 7. The sterilizable cover 228 may cover the housing 222 and/or portions of the stimulator element 224 to create a sterile instrument. The cover 228 may be disposable or may be re-usable and sterilized between uses. In an illustrative embodiment, the cover 228 is clear and allows a user to see and activate the controls on the housing 222. In other illustrative embodiments, only portions of the cover 228 overlying controls on the housing 222 may be clear. The cover 228 may be made of plastic, cloth, or any other sterilizable material.

While a number of different controls and displays are described, any number of such controls or displays may be used and/or any number of additional controls or displays may be used.

1. A bipolar cortical stimulator, comprising:
(a) a handheld housing including one or more batteries for powering the bipolar cortical stimulator;
(b) a stimulator element extending from the housing;
(c) a circuit board disposed within the housing for controlling operation of the stimulator element and powered by the one or more batteries;
(d) a plurality of controls extending from the housing and operatively connected to the circuit board to control one or more parameters of the stimulator element; and
(e) a digital display disposed within the housing for displaying the one or more parameters of the stimulator element.

2. The bipolar cortical stimulator of claim 1, further including a sterilizable cover disposed over and covering the housing.

3. The bipolar cortical stimulator of claim 2, wherein the sterilizable cover is disposable.

4. The bipolar cortical stimulator of claim 2, wherein the sterilizable cover covers at least a portion of the stimulator element.

5. The bipolar cortical stimulator of claim 1, wherein at least one of the plurality of controls is selected from the group consisting of a control allowing an increase in duration, a control allowing a decrease in duration, a control allowing an increase in frequency, a control allowing a decrease in frequency, a control allowing an increase in current, and a control allowing a decrease in current.

6. The bipolar cortical stimulator of claim 1, wherein the plurality of controls includes a control allowing an increase in duration, a control allowing a decrease in duration, a control allowing an increase in frequency, a control allowing a decrease in frequency, a control allowing an increase in current, and a control allowing a decrease in current.

7. The bipolar cortical stimulator of claim 6, wherein the digital display includes a first display for displaying a current or selected duration, a second display for displaying a current or selected frequency, and a third display for displaying a current or selected current.

8. The bipolar cortical stimulator of claim 1, further comprising an electroencephalograph.

9. The bipolar cortical stimulator of claim 8, wherein the electroencephalograph is operatively connected to the circuit board and records electrical activity from the stimulator element.

10. The bipolar cortical stimulator of claim 10, wherein the electroencephalograph is configured to record electrical activity when current exerted by the bipolar cortical stimulator across the stimulator element is reduced to about 0 milliamps.

11. The bipolar cortical stimulator of claim 10, further comprising an additional controller extending from the housing and operatively connected to the circuit board allowing the bipolar cortical simulator to switch between a cortical stimulating mode by turning the stimulator element on and an electroencephalogram recording mode by turning the stimulator element off.

12. The bipolar cortical stimulator of claim 10, further comprising a digital electroencephalogram display disposed within the housing for displaying an electroencephalogram recorded by the electroencephalograph.

13. The bipolar cortical stimulator of claim 10, further comprising a seizure warning element wherein the seizure warning element is operatively connected to the circuit board and configured to convey a warning when a single recorded electroencephalogram or a series of recorded electroencephalograms contain an afterdischarge.

14. A bipolar cortical stimulator, comprising:
(a) a handheld housing including one or more batteries for powering the bipolar cortical stimulator;
(b) a stimulator element extending from the housing;
(c) a circuit board disposed within the housing for controlling operation of the stimulator element and powered by the one or more batteries; and
(d) a control extending from the housing and operatively connected to the circuit board to control an increase in current or a decrease in current of the stimulator element.

15. A method of stimulating cortex in a patient using the bipolar cortical stimulator of claim 1, the method comprising:
(a) holding the housing of the bipolar cortical stimulator with a first hand of a user;
(b) placing the stimulator element adjacent the cortex of the patient; and
(c) operating the plurality .of controls with a second hand of the user to change the one or more parameters of the stimulator element.

16. The method of claim 15, further including the step of reading the plurality of controls and simultaneously operating the plurality of controls to further change the one or more parameters of the stimulator element.

17. A method of stimulating cortex in a patient using the bipolar cortical stimulator of claim 10, the method comprising:
(a) holding the housing of the bipolar cortical stimulator with a first hand of a user;
(b) placing the stimulator element adjacent the cortex of the patient; and
(c) operating the plurality of controls with a second hand of the user to change the one or more parameters of the stimulator element;

18. The method of claim 17, further including the step of monitoring the electroencephalogram of the patient for a sign of a seizure.

19. The method of claim 18, further including the step of reading the plurality of controls and simultaneously operating the plurality of controls to further change the one or more parameters of the stimulator element.
